(19) United States
(12) Patent Application Publication
(10) Pub. No.: US 2026/0115197 A1
(43) Pub. Date: Apr. 30, 2026

(54) TREATMENT COMPOSITION FOR INHIBITING SYSTEMIC SCLEROSIS VIMENTIN MUTANT PROTEIN ACTIVITY BY USING STAT6 INHIBITOR

(71) Applicants: THE CATHOLIC UNIVERSITY OF KOREA INDUSTRY-ACADEMIC COOPERATION FOUNDATION, Seoul (KR); GACHON UNIVERSITY OF INDUSTRY-ACADEMIC COOPERATION FOUNDATION, Seongnam-si, Gyeonggi-do (KR)

(72) Inventors: Mi La CHO, Seoul (KR); Seon Yeong LEE, Seoul (KR); Kun-Hee LEE, Seoul (KR); Chae-Rim LEE, Seoul (KR); Su-Been JEON, Hwaseong-si (KR); Dong Yun SHIN, Seoul (KR); Pun RABIN, Incheon (KR); Aram LEE, Jinju-si (KR)

(73) Assignees: THE CATHOLIC UNIVERSITY OF KOREA INDUSTRY-ACADEMIC COOPERATION FOUNDATION, Seoul (KR); GACHON UNIVERSITY OF INDUSTRY-ACADEMIC COOPERATION FOUNDATION, Seongnam-si, Gyeonggi-do (KR)

(21) Appl. No.: 19/116,721

(22) PCT Filed: Nov. 17, 2023
(86) PCT No.: PCT/KR2023/018585

§ 371 Date: Mar. 28, 2025

(30) Foreign Application Priority Data
Nov. 18, 2022 (KR) 10-2022-0155044
Nov. 17, 2023 (KR) 10-2023-0159686

(51) Int. Cl. A61K 31/5377; A61K 45/00; A61P 29/00; A61P 37/02

(52) U.S. Cl. CPC A61K 31/5377; A61K 45/00; A61P 29/00; A61P 37/02

(57) ABSTRACT

A treatment composition for inhibiting systemic sclerosis Vimentin mutant protein activity by using an STAT6 inhibitor, which inhibits the expression of an M2 macrophage and a profibrotic T cell which are immunocytes related to systemic sclerosis, and increases the expression of a Treg, and inhibits the expression of TGF-β, Col1a1, and α-SMA which are fibrosis factors related to systemic sclerosis. The presence of a pSTAT6 expression CD8 T cell in a fibrosis tissue has been identified, and that the expression of a pSTAT6 expression CD8 T positive cell is controlled via injection of the STAT6 inhibitor. In an animal model with increased Vimentin-specific disease symptom activity, the STAT6 inhibitor inhibits antigen-specific tissue fibrosis of systemic sclerosis with activated disease symptoms, and that the STAT6 inhibitor inhibits the expression of IL-17 cytokine expression CD8 positive TRM capable of inducing fibrosis and cell inflammation which are systemic sclerosis diseases.

### CROSS REFERENCE TO RELATED APPLICATIONS

This application is a National Stage of International Application No. PCT/KR2023/018585 filed Nov. 17, 2023, claiming priority based on Korean Patent Application No. 10-2022-0155044 filed Nov. 18, 2022 and Korean Patent Application No. 10-2023-0159686 filed Nov. 17, 2023, the entire disclosures of which are incorporated herein by reference.

### TECHNICAL FIELD

The present disclosure relates to a treatment composition for inhibiting systemic sclerosis Vimentin mutant protein activity by using a STAT6 inhibitor.

### BACKGROUND ART

Diseases caused by immune hypersensitivity are increasing worldwide, but the fundamental causes of these diseases have not been sufficiently identified. A current treatment method for diseases caused by excessive immune responses is to alleviate or reduce various symptoms caused by the diseases by administering immunosuppressants alone or in combination.

The immunosuppressants refer to various substances used to reduce or block the ability (humoral immune response) of preparing antibodies by a host or the ability of causing a cellular immune response in the action of an antigen. These immunosuppressants may be useful not only in the field of organ transplantation, but also in autoimmune diseases such as lupus and rheumatoid arthritis, and skin hypersensitivity such as atopy and allergies. An excellent immunosuppressant should be able to control the imbalance of the immune responses, ensure safety for the human body, and have a low incidence of disease recurrence during long-term treatment.

Currently used immunosuppressants include cyclosporin A, FK506, etc., which are natural product-derived compounds with complex chemical structures, and are uneconomical due to a problem of high cost in terms of raw material supply, so that there is a risk of causing various side effects due to long-term administration. Accordingly, there is an urgent need for the development of new immunosuppressants that are able to be economically produced with low toxicity and immune tolerance induction.

Meanwhile, Vimentin is a structural protein encoded by the VIM gene in humans, and known to play an important role in supporting and anchoring the positions of organelles. The cytoskeleton of chondrocytes is mainly composed of actin microfilaments, tubulin microtubules, and Vimentin intermediate filaments. Vimentin is known to be important for the structural formation of cells and tissues, but in a patient or animal model with autoimmune diseases, the protein expression level and the filament network organization of Vimentin were changed to identify abnormal overexpression of Vimentin, which was reported to be related to a damaged Vimentin network. In addition, systemic sclerosis and tissue fibrosis are caused by chronic inflammation related to repetitive tissue damage and autoimmune diseases, and when a conventional tissue repair system is interrupted, chronic inflammation is induced, and inflammatory cytokines, chemokines, and growth factors are secreted from immunocytes. These factors induce the activation of fibroblasts and astrocytes, and induce transformation into myofibroblasts and activated astrocytes, respectively. Thereafter, the activated myofibroblasts and astrocytes induce the production of components of the extracellular matrix, such as collagen, and the corresponding tissue is hardened due to excessive accumulation of the extracellular matrix to cause structural and functional damage. At this time, it was reported that cell surface Vimentin was specifically expressed in the activated myofibroblasts and astrocytes.

Therefore, there is a need to develop a new therapeutic agent capable of alleviating systemic sclerosis by specifically acting on Vimentin.

### DISCLOSURE

### Technical Problem

An object of the present disclosure is to provide a pharmaceutical composition for preventing or treating systemic sclerosis, including a signal transducer and activator of transcription 6 (STAT6) inhibitor as an active ingredient.

Another object of the present disclosure is to provide a food composition for preventing or alleviating systemic sclerosis, including a STAT6 inhibitor as an active ingredient.

Yet another object of the present disclosure is to provide a quasi-drug composition for preventing or alleviating systemic sclerosis, including a STAT6 inhibitor as an active ingredient.

Yet another object of the present disclosure is to provide a pharmaceutical composition for preventing or treating fibrosis by a Vimentin mutant protein, including a STAT6 inhibitor as an active ingredient.

Yet another object of the present disclosure is to provide a treatment method of systemic sclerosis including administering a STAT6 inhibitor in a pharmaceutically effective amount to a subject.

Yet another object of the present disclosure is to provide a treatment method of fibrosis by a Vimentin mutant protein, including administering a STAT6 inhibitor in a pharmaceutically effective amount to a subject.

### Technical Solution

In order to achieve the objects, an aspect of the present disclosure provides a pharmaceutical composition for preventing or treating systemic sclerosis, including a signal transducer and activator of transcription 6 (STAT6) inhibitor as an active ingredient.

Another aspect of the present disclosure provides a food composition for preventing or alleviating systemic sclerosis, including a STAT6 inhibitor as an active ingredient.

Yet another aspect of the present disclosure provides a quasi-drug composition for preventing or alleviating systemic sclerosis, including a STAT6 inhibitor as an active ingredient.

Yet another aspect of the present disclosure provides a pharmaceutical composition for preventing or treating fibrosis by a Vimentin mutant protein, including a STAT6 inhibitor as an active ingredient.

Yet another aspect of the present disclosure provides a treatment method of systemic sclerosis, including administering a STAT6 inhibitor in a pharmaceutically effective amount to a subject.

Yet another aspect of the present disclosure provides a treatment method of fibrosis by a Vimentin mutant protein, including administering a STAT6 inhibitor in a pharmaceutically effective amount to a subject.

### Advantageous Effects

According to the present disclosure, it has been identified that an STAT6 inhibitor inhibits the expression of a profibrotic T cell and an M2 macrophage which are immunocytes related to systemic sclerosis, and increases the expression of Treg, and inhibits the expression of TGF-β, Col1a1, and α-SMA which are fibrosis factors related to systemic sclerosis. In addition, it has been identified that when a STAT6 inhibitor was administered to an animal model of systemic sclerosis, the STAT6 inhibitor inhibits the activity of an M2 macrophage capable of inducing fibrosis in a mouse blood, and suppresses the thickness of the skin dermis and pulmonary fibrosis. It has been identified that the expression of cell surface Vimentin, which is a Vimentin mutant protein related to systemic sclerosis, is reduced in a skin tissue and a lung tissue, and that the Vimentin mutant protein can be controlled. In addition, the presence of a pSTAT6-expressing CD8 T cell in a fibrosis tissue has been identified, and it has been identified that the expression of a pSTAT6-expressing CD8 T positive cell is controlled via injection of the STAT6 inhibitor. Even in an animal model with increased Vimentin-specific disease symptom activity, the STAT6 inhibitor inhibits antigen-specific tissue fibrosis of systemic sclerosis with activated disease symptoms, and inhibits the expression of IL-17 cytokine-expressing CD8 positive TRM capable of inducing fibrosis and inflammation, which is a pathogenic cell of systemic sclerosis, and thus can be effectively used in related industries.

### DESCRIPTION OF DRAWINGS

FIG. 1 is a diagram identifying the control of immune cells in splenocytes according to treatment with a STAT6 inhibitor of the present disclosure (A: identification of profibrotic T cell expression, B: identification of M2 macrophage expression, C: identification of Treg expression, D: quantification of M2/M1 expression ratio).

FIG. 2 is a diagram identifying the expression of fibrosis genes in fibroblasts according to treatment with a STAT6 inhibitor of the present disclosure (A: identification of TGF-β expression, B: identification of Col1a1 expression, C: identification of α-SMA expression).

FIG. 3 is a diagram identifying the inhibition of M2 macrophage activity in the blood according to administration of a STAT6 inhibitor in a systemic sclerosis mouse by flow cytometry.

FIG. 4 is a diagram identifying a dermal layer thickness according to administration of a STAT6 inhibitor in a systemic sclerosis mouse using H&E staining (A: staining result, B: quantification of staining result).

FIG. 5 is a diagram identifying pulmonary fibrosis according to administration of a STAT6 inhibitor in a systemic sclerosis mouse using H&E staining (A: staining result, B: quantification of staining result).

FIG. 6 is a diagram identifying the expression of fibrosis factors and cell surface Vimentin in a skin tissue according to administration of a STAT6 inhibitor in a systemic sclerosis mouse using immunohistochemical staining (A: staining result, B: quantification of staining result).

FIG. 7 is a diagram identifying the expression of fibrosis factors and cell surface Vimentin in a lung tissue according to administration of a STAT6 inhibitor in a systemic sclerosis mouse using immunohistochemical staining (A: staining result, B: quantification of staining result).

FIG. 8 is a diagram identifying the expression of pSTAT6 and CD8 double positive cells in skin and lung tissues according to administration of a STAT6 inhibitor in a systemic sclerosis mouse using confocal microscopy (A: confocal analysis result, B: quantification of analysis result).

FIG. 9 is a schematic diagram illustrating construction of a Vimentin antigen-specific systemic sclerosis hyperactivation animal model and a drug administration schedule.

FIG. 10 is a diagram identifying skin fibrosis and the expression of cell surface Vimentin in a skin tissue according to treatment of a STAT6 inhibitor in a Vimentin antigen-specific systemic sclerosis hyperactivation animal model using H&E staining and immunohistochemical staining (A: staining result, B: quantification of skin thickness, C: quantification of cell surface Vimentin).

FIG. 11 is a diagram identifying the expression of pathogenic IL-17-expressing CD8 TRM (CD103 and CD8 double positive) according to treatment with a STAT6 inhibitor in a Vimentin antigen-specific systemic sclerosis hyperactivation animal model using cofocal microscopy.

### BEST MODE OF THE INVENTION

Hereinafter, examples of the present disclosure will be described in detail with reference to the accompanying drawings. In the following description, detailed descriptions of techniques well-known to those skilled in the art may be omitted. Further, in describing the present disclosure, the detailed description of associated known functions or constitutions will be omitted if it is determined to unnecessarily make the gist of the present disclosure unclear. Further, terminologies used in the present disclosure are terminologies used to properly express preferred exemplary embodiments of the present disclosure, which may vary according to a user, an operator's intention, or customs in the art to which the present disclosure pertains.

Accordingly, definitions of the terminologies need to be described based on contents throughout this specification. Throughout the specification, unless explicitly described to the contrary, when a certain part “comprises” a certain component, it will be understood to imply the inclusion of stated elements but not the exclusion of any other elements.

The present disclosure provides a pharmaceutical composition for preventing or treating systemic sclerosis, including a signal transducer and activator of transcription 6 (STAT6) inhibitor as an active ingredient.

As used in the present disclosure, the term “prevention” refers to all actions that suppress the symptoms of a specific disease or delays its progression by administering the composition of the present disclosure.

As used in the present disclosure, the term “treatment” refers to all actions that alleviate or beneficially change the symptoms of a specific disease by administering the composition of the present disclosure.

The pharmaceutical composition of the present disclosure may further include an adjuvant in addition to the active ingredient. The adjuvant may be used with any adjuvant known in the art without limitation, but further include, for example, a Freund's complete adjuvant or an incomplete adjuvant to increase the effect thereof.

The pharmaceutical composition according to the present disclosure may be prepared in the form of incorporating the active ingredient into a pharmaceutically acceptable carrier. Here, the pharmaceutically acceptable carrier includes carriers, excipients and diluents commonly used in a pharmaceutical field. The pharmaceutically acceptable carrier that may be used in the pharmaceutical composition of the present disclosure is not limited thereto, but may include lactose, dextrose, sucrose, sorbitol, mannitol, xylitol, erythritol, maltitol, starch, acacia gum, alginate, gelatin, calcium phosphate, calcium silicate, cellulose, methyl cellulose, polyvinylpyrrolidone, water, methyl hydroxybenzoate, propyl hydroxybenzoate, talc, magnesium stearate, and mineral oil.

The pharmaceutical composition of the present disclosure may be formulated and used in the form of oral formulations, such as powders, granules, tablets, capsules, suspensions, emulsions, syrups, aerosols, etc., external preparations, suppositories, and sterile injectable solutions according to each conventional method.

The formulations may be prepared by using diluents or excipients, such as a filler, an extender, a binder, a wetting agent, a disintegrating agent, a surfactant, etc., which are generally used. Solid formulations for oral administration include tablets, pills, powders, granules, capsules, etc., and these solid formulations may be prepared by mixing at least one or more excipients, for example, starch, calcium carbonate, sucrose, lactose, gelatin, etc. with the active ingredients. Further, lubricants such as magnesium stearate and talc may be used in addition to simple excipients. Liquid formulations for oral administration may correspond to suspensions, oral liquids, emulsions, syrups, etc., and may include various excipients, for example, a wetting agent, a sweetener, an aromatic agent, a preserving agent, etc., in addition to the commonly used diluents, such as water and liquid paraffin. Formulations for parenteral administration include sterile aqueous solutions, non-aqueous solvents, suspensions, emulsions, lyophilized agents, and suppositories. As the non-aqueous solution and the suspension, propylene glycol, polyethylene glycol, vegetable oil such as olive oil, injectable ester such as ethyl oleate, etc. may be used. As the base material of the suppository, witepsol, Tween 61, cacao butter, laurinum, glycerogelatin, etc. may be used.

The pharmaceutical composition according to the present disclosure may be administered to a subject through various routes. All methods of administration may be expected, and the pharmaceutical composition may be administered, for example, oral, intravenous, intramuscular, subcutaneous, and intraperitoneal injection.

The dose of the pharmaceutical composition according to the present disclosure is selected in consideration of the age, body weight, sex, and physical conditions of the subject. It is obvious that the concentration of the active ingredients included in the pharmaceutical composition may be variously selected according to a subject, and preferably included in the pharmaceutical composition at a concentration of 0.01 to 5,000 μg/ml. When the concentration is less than 0.01 μg/ml, pharmaceutical activity may not be shown, and when the concentration exceeds 5,000 μg/ml, toxicity to the human body may be exhibited.

The “signal transducer and activator of transcription 6 (STAT6)” of the present disclosure is a STAT-based transcription factor, and STAT-based proteins transmit signals from a receptor complex to a nucleus and activate gene expression. Similarly to other STAT-based proteins, STAT6 is also activated by growth factors and cytokines, and STAT6 is known to be mainly activated by IL-4 and IL-13. A STAT6 signaling pathway is required for the development of Th2 cells and Th2 immune responses, and activation of STAT6 signaling is known to be essential for the function of macrophages and required for activation of subtypes of the M2 macrophages.

According to an example of the present disclosure, the STAT6 inhibitor may be a compound represented by Chemical Formula 1 below or a pharmaceutically acceptable salt thereof.

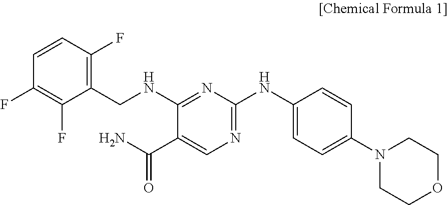 [SMILES: NC(=O)c1cnc(Nc2ccc(N3CCOCC3)cc2)nc1NCc1c(F)ccc(F)c1F]

According to an example of the present disclosure, the STAT6 inhibitor may further include a substance that specifically binds to a STAT6 gene to inhibit the gene expression of STAT6, or a substance that specifically binds to a STAT6 protein to inhibit the expression or activity of the STAT6 protein. The STAT6 gene expression inhibitor may be selected from the group consisting of antisense nucleotide, siRNA, and shRNA that complementarily bind to mRNA of the STAT6 gene. The STAT6 protein expression or activity inhibitor may be selected from the group consisting of a peptide, a peptidomimetic, a substrate analog, an aptamer, and an antibody that complementarily binds to the STAT6 protein, but is not limited thereto as long as the inhibitor is a substance that inhibits the expression or activity of the STAT6 gene or protein.

According to an example of the present disclosure, the systemic sclerosis may further include systemic sclerosis with increased disease symptom activity by Vimentin.

According to an example of the present disclosure, the STAT6 inhibitor may reduce a skin thickness.

According to an example of the present disclosure, the STAT6 inhibitor may inhibit tissue fibrosis induced by systemic sclerosis, and the tissue may be a tissue selected from the group consisting of skin, lung, liver, muscle, kidney, intestine, and spleen, but is not limited thereto.

According to an example of the present disclosure, the STAT6 inhibitor may control immunocytes in the tissue, spleen or blood.

According to an example of the present disclosure, the controlling of the immunocytes may be reducing the expression of a profibrotic T cell or M2 macrophage, reducing the expression ratio of a M2 macrophage and a M1 macrophage, and reducing the activity of the M2 macrophage.

According to an example of the present disclosure, the controlling of the immunocytes may be increasing the expression of Treg.

According to an example of the present disclosure, the controlling of the immunocytes may be reducing the expression of pSTAT6 positive and CD8 positive cells: or IL-17 positive, CD103 positive and CD8 positive cells.

According to an example of the present disclosure, the STAT6 inhibitor may reduce fibrosis factors, and the fibrosis factors may be factors selected from the group consisting of TGF-β, Col1a1, α-SMA, and IL-17.

According to an example of the present disclosure, the STAT6 inhibitor may inhibit the activity of a Vimentin mutant protein in a tissue, and the Vimentin mutant protein may be cell surface Vimentin.

The “Vimentin mutant protein” of the present disclosure is cell surface Vimentin, which is Vimentin expressed on the surface when myofibroblasts and astrocytes in a fibrosis tissue are activated, and it has been reported that the activated myofibroblasts and astrocytes secrete collagen or extracellular matrix within tissue cells to harden the tissue and induce tissue fibrosis. In addition, under activation and oxidative stress environments of inflammatory cytokines, filamentous Vimentin is modified by citrullination, phosphorylation, and the like, and the modified Vimentin loses the function as a filament and increases the production of an autoantibody as an autoantigen. In addition, fragmented Vimentin or modified Vimentin may induce the activity of a macrophage and other cells as an autoantigen

Further, the present disclosure provides a food composition for preventing or alleviating systemic sclerosis, including a STAT6 inhibitor as an active ingredient.

As used in the present disclosure, the term “improvement” means all actions that at least reduce parameters related to conditions to be treated, such as the degree of symptoms.

In addition to containing the active ingredient of the present disclosure, the food composition of the present disclosure may contain various flavoring agents or natural carbohydrates as an additional ingredient, like conventional food compositions.

Examples of the above-described natural carbohydrates include conventional sugars, including monosaccharides, such as glucose, fructose, etc.; disaccharides, such as maltose, sucrose, etc.; and polysaccharides, such as dextrin, cyclodextrin, etc., and sugar alcohols such as xylitol, sorbitol, erythritol, etc. The above-described flavoring agents may be advantageously used with natural flavoring agents (thaumatin), stevia extracts (e.g., rebaudioside A, glycyrrhizin, etc.), and synthetic flavoring agents (saccharin, aspartame, etc.). The food composition of the present disclosure may be formulated in the same manner as the pharmaceutical composition to be used as a functional food or added to various foods. The foods capable of adding the composition of the present disclosure include, for example, beverages, meat, chocolate, foods, confectionery, pizza, ramen, other noodles, gums, candies, ice creams, alcohol beverages, vitamin complexes, health food supplements, etc.

In addition, the food composition may contain various nutrients, vitamins, minerals (electrolytes), flavoring agents such as synthetic and natural flavoring agents, coloring agents and enhancers (cheese, chocolate, etc.), pectic acid and salts thereof, alginic acid and salts thereof, organic acid, a protective colloidal thickener, a pH adjusting agent, a stabilizer, a preservative, glycerin, alcohol, a carbonic acid agent used in a carbonated drink, and the like, in addition to the extract as the active ingredient. In addition, the food composition of the present disclosure may contain pulps for preparing natural fruit juice, fruit juice beverages, and vegetable beverages.

The functional food composition of the present disclosure may be prepared and processed in the form of tablets, capsules, powders, granules, liquids, pills, etc. for the purpose of prevention or treatment of systemic sclerosis. In the present disclosure, the ‘health functional food composition’ refers to foods prepared and processed by using raw materials or ingredients with functionality, which are useful for the human body according to the Art on Health Functional Foods No. 6727, and means foods taken for adjusting nutrients for the structures and functions of the human body or obtaining a useful effect on health applications such as physiological actions. The health functional food of the present disclosure may include conventional food additives, and the suitability as the food additives is determined by the specifications and standards for the corresponding item in accordance with the general rules of the Food Additives Codex, general test methods, etc., that are approved by the Food and Drug Administration, unless otherwise specified. The items disclosed in the ‘Food Additives Codex’ may include, for example, chemical composites such as ketones, glycine, calcium citrate, nicotinic acid, cinnamic acid, etc.: natural additives such as persimmon color, licorice extract, crystal cellulose, Kaoliang color, guar gum, etc.; mixed formulations such as sodium L-glutamic acid formulations, noodle additive alkali agents, preservative formulations, tar color formulations, etc. For example, the health functional food in the form of tablets may be formed by granulating a mixture obtained by mixing the active ingredient of the present disclosure with an excipient, a binder, a disintegrant, and other additives by conventional method, and then compression-molding the mixture by adding a slip modifier and the like, or directly compressing the mixture. In addition, the health functional food in the form of tablets may also contain a flavors enhancer or the like as needed. In the health functional food in the form of capsules, hard capsules may be prepared by filling a mixture mixed with the active ingredient of the present disclosure and additives such as excipients into conventional hard capsules, and soft capsules may be prepared by filling a mixture mixed with the active ingredient of the present disclosure and additives such as excipients into capsule bases such as gelatin. The soft capsules may contain a plasticizer such as glycerin or sorbitol, a colorant, a preservative, and the like, if necessary. The health functional food in the form of pills may be prepared by molding a mixture obtained by mixing the active ingredient of the present disclosure with an excipient, a binder, a disintegrant, etc. by conventional known methods, and may also be coated with white sugar or other coating agents or surface-coated with materials such as starch and talc, if necessary. The health functional food in the form of granules may be prepared by granulizing a mixture obtained by mixing the active ingredient of the present disclosure with an excipient, a binder, a disintegrant, etc. by conventional known methods and may contain a flavoring agent, a flavors enhancer, etc., if necessary.

Further, the present disclosure provides a quasi-drug composition for preventing or alleviating systemic sclerosis, including a STAT6 inhibitor as an active ingredient.

As used in the present disclosure, the term “quasi-drug” refers to products with a milder action than medicines among products used for the purpose of diagnosing, treating, alleviating, mitigating, handling or preventing human or animal diseases. For example, according to the Pharmaceutical Affairs Act, the quasi-drug includes products used for the treatment or prevention of human/animal diseases, products with mild or no direct action on the human body, etc., by excluding products used for the purpose of medicines.

The quasi-drug composition of the present disclosure may be prepared in a formulation selected from the group consisting of a body cleanser, a disinfectant cleaner, a detergent, a kitchen detergent, a cleaning detergent, a toothpaste, a gargle, a wet tissue, a detergent, soap, a hand wash, a hair cleanser, a hair softener, a humidifier filler, a mask, an ointment, and a filter filler, but is not limited thereto.

Further, the present disclosure provides a pharmaceutical composition for preventing or treating fibrosis by a Vimentin mutant protein, including a STAT6 inhibitor as an active ingredient.

Further, the present disclosure provides a treatment method of systemic sclerosis, including administering a STAT6 inhibitor in a pharmaceutically effective amount to a subject.

The treatment method of the present disclosure includes administering to a subject the compound represented by Chemical Formula 1 or a pharmaceutically acceptable salt thereof in a therapeutically effective amount. It is preferred that a specific therapeutically effective amount for a specific subject is differently applied depending on various factors including the type and degree of a response to be achieved, a specific composition including whether other agents are used in some cases, the age, body weight, general health conditions, sex, and diet of a subject, an administration time, an administration route, a secretion rate of the composition, a duration of treatment, and a drug used in combination or simultaneously with the specific composition, and similar factors well known in the medical field. A daily dose may be 0.0001 to 100 mg/kg, preferably 0.01 to 100 mg/kg, based on the amount of the pharmaceutical composition of the present disclosure, and may be administered 1 to 6 times a day. However, it is obvious to those skilled in the art that the dosage or dose of each active ingredient does not cause side effects by including the content of each active ingredient too high. Therefore, the effective amount of the composition suitable for the purpose of the present disclosure is preferably determined in consideration of the aforementioned matters.

The subject is applicable to any mammal, and the mammal includes not only humans and primates, but also livestock such as cattle, pig, sheep, horse, dog, and cat.

The compound represented by Chemical Formula 1 of the present disclosure or the pharmaceutically acceptable salt thereof may be administered to mammals such as mice, rats, livestock, and humans through various routes. All methods of administration may be expected, and for example, the pharmaceutical composition may be administered by oral, rectal or intravenous, intramuscular, subcutaneous, intrauterine dural or intracerebroventricular injection.

Further, the present disclosure provides a treatment method of fibrosis by a Vimentin mutant protein, including administering a STAT6 inhibitor in a pharmaceutically effective amount to a subject.

### Modes of the Invention

Hereinafter, the present disclosure will be described in more detail through Examples. These Examples are to explain the present disclosure in more detail, and it will be apparent to those skilled in the art that the scope of the present disclosure is not limited to these Examples.

### <Example 1> Identification of Control of Immunocytes Related to Fibrosis by STAT6 Inhibition

In order to identify an effect of inhibiting signal transducer and activator of transcription 6 (STAT6) on systemic sclerosis of the present disclosure, the control effect of immunocytes related to fibrosis was identified. Specifically, spleen single cells were obtained from a B6 mouse, treated with 10 ng/ml of transforming growth factor-β (TGF-β) and 10 nM of a STAT6 inhibitor (YM-341619, Chemical Formula 1), and then cultured for 72 hours. The expression of profibrotic T cells, M2 macrophages, and Treg related to systemic sclerosis was analyzed by flow cytometry. In addition, the expression ratio of M2 macrophages and M1 macrophages was identified to identify the control of immunocytes. As a control group, an untreated control group (Nil) and a group treated with TGF-β alone were used.

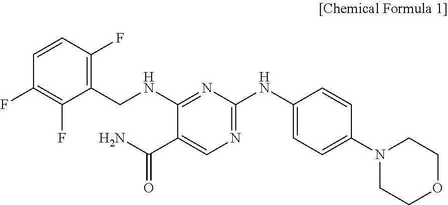 [SMILES: NC(=O)c1cnc(Nc2ccc(N3CCOCC3)cc2)nc1NCc1c(F)ccc(F)c1F]

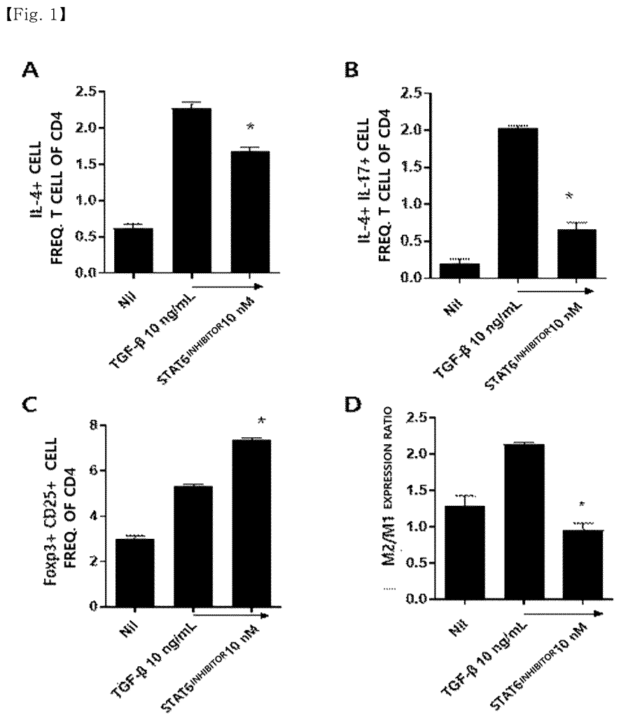

As a result, as illustrated in FIG. 1, it has been identified that in the group treated with TGF-β alone, the expression of pro-fibrosis T cells (IL-4+ T cells and IL-4+ and IL-17+ T cells) and M2 macrophages increased compared to the Nil group, but in a group treated with a STAT6 inhibitor, the increased expression of pro-fibrosis T cells and M2 macrophages was significantly reduced (FIGS. 1A and 1B). In addition, it has been identified that the expression of Treg was significantly increased in the group treated with the STAT6 inhibitor, compared to the group treated with TGF-β alone (FIG. 1C). In addition, it has been identified that in the group treated with TGF-β alone, the M2/M1 expression ratio increased compared to the Nil group, but the M2/M1 expression ratio increased by TGF-β was significantly reduced by treatment with the STAT6 inhibitor (FIG. 1D), and thus it has been identified that the STAT6 inhibitor controlled the immunocytes related to systemic sclerosis.

### <Example 2> Identification of the Inhibition of the Expression of Fibrosis Factors by STAT6 Inhibition

In order to identify the effect of STAT6 inhibition on systemic sclerosis of the present disclosure, inhibition of fibrosis genes of fibroblasts was identified. Specifically, a human dermal fibroblast line was treated with 10 ng/ml of TGF-β and nM of an STAT6 inhibitor and then cultured for 24 hours. Thereafter, the expression of fibrosis factors, TGF-β, Col1a1 (Collagen, type I, alpha 1), and α-SMA 10 (alpha smooth muscle actin), was identified by real-time PCR.

As a result, as illustrated in FIG. 2, it has been identified that compared to the Nil group, in the group treated with TGF-β alone, the expression of fibrosis genes in fibroblasts, TGF-β, Col1a1, and α-SMA, significantly increased, but when treated with the STAT6 inhibitor, the increased expression of fibrosis genes significantly decreased.

### <Example 3> Identification of Control of M2 Macrophage Activity by STAT6 Inhibition in Systemic Sclerosis Animal Model

To determine whether the STAT6 inhibitor of the present disclosure alleviated systemic sclerosis, a systemic sclerosis animal model was constructed. Specifically, a 2×2 cm area on the nape of the neck of the B6 mouse was shaved using a clipper, and then subcutaneously injected with 100 μL of PBS containing 50 μg of bleomycin (BLM) daily for 2 weeks to induce systemic sclerosis. One week after the start of BLM administration, 200 μL of vehicle solution (10% DMSO, 40% PEG300, 5% tween 80 and 45% Saline) containing the STAT6 inhibitor at a concentration of 10 mg/kg was orally administered daily. Thereafter, the blood from the mouse was collected on week 1 and week 5 after induction of systemic sclerosis, and the activity of M2 macrophages capable of inducing fibrosis from the collected blood was analyzed using flow cytometry. As a control group, a wild-type mouse (WT) and a Vehicle group that was induced with systemic sclerosis but not treated with a drug were used.

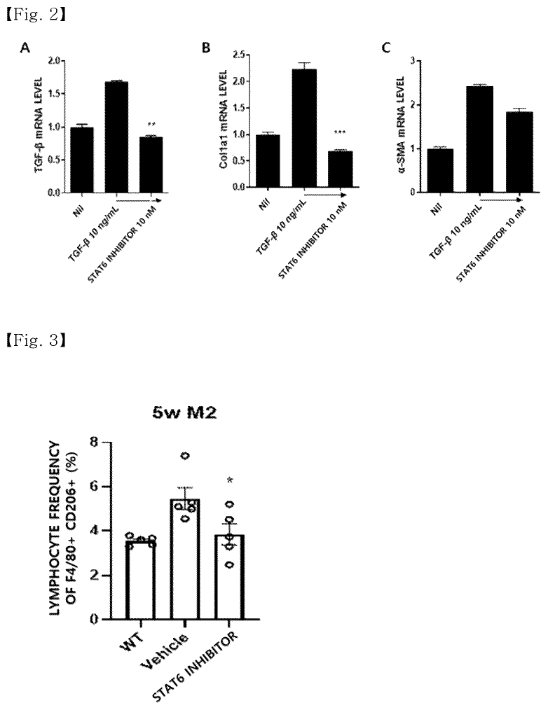

As a result, as illustrated in FIG. 3, it has been identified that compared to the wild-type mouse, the activity of M2 macrophages capable of inducing fibrosis was significantly increased in the Vehicle group, but in the group administered with the STAT6 inhibitor, the increased activity of M2 macrophages capable of inducing fibrosis was significantly reduced, and thus it has been identified that the STAT6 inhibitor may alleviate systemic sclerosis.

### <Example 4> Identification of Inhibition of Tissue Fibrosis by STAT6 Inhibition in Systemic Sclerosis Animal Model

### <4-1> Identification of Inhibition of Skin Fibrosis

To determine whether the STAT6 inhibitor of the present disclosure alleviated systemic sclerosis, inhibition of skin fibrosis was identified. Specifically, the mouse of Example 3 was humanely sacrificed at the end point of the experiment, and skin tissue was obtained, and then subjected to hematoxylin & eosin (H&E) staining to identify the effect of controlling skin inflammation responses and fibrosis by a change in skin thickness.

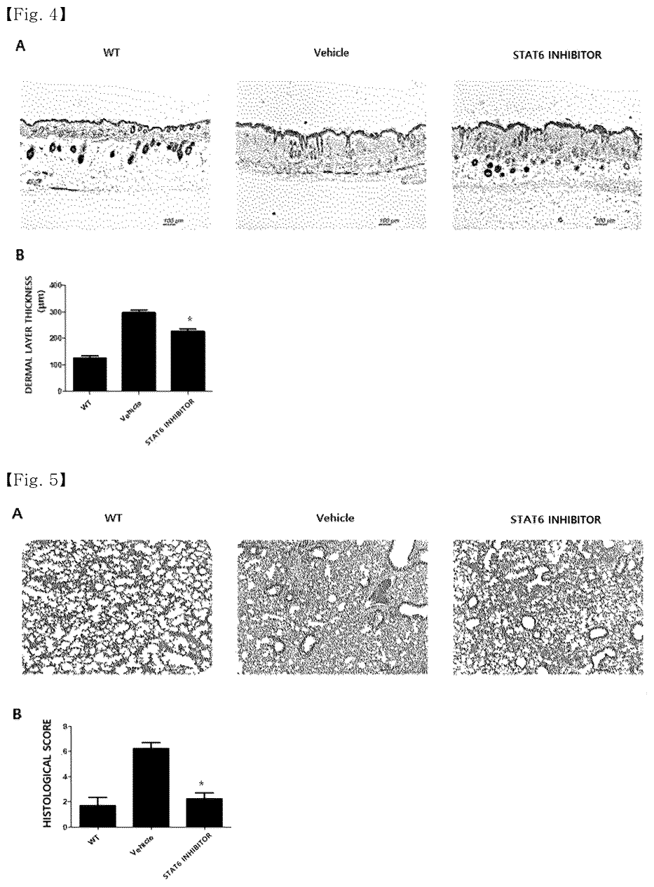

As a result, as illustrated in FIG. 4, it has been identified that compared to the wild-type mouse, the thickness of the skin dermal layer in the Vehicle group was significantly increased, but the increased thickness of the dermal layer was significantly reduced by administration of the STAT6 inhibitor.

### <4-2> Identification of Inhibition Effect of Pulmonary Fibrosis

It has been identified whether the STAT6 inhibition of the present disclosure inhibited pulmonary fibrosis in systemic sclerosis. Specifically, a lung tissue was obtained from the mouse sacrificed in the Example 4-1 above, and subjected to H&E staining to determine the histological score for pulmonary fibrosis.

As a result, as illustrated in FIG. 5, it has been identified that compared to the wild-type mouse, the histological score was significantly increased in the Vehicle group due to increased lung inflammation and fibrosis, but the increased histological score was significantly reduced when the STAT6 inhibitor was administered.

### <Example 5> Identification of Expression Inhibition of Fibrosis Factors and Cell Surface Vimentin in Systemic Sclerosis

### <5-1> Identification of Expression Inhibition of Fibrosis Factors and Cell Surface Vimentin in Skin Tissue

In autoimmune diseases, it has been reported that Vimentin played an important role in cell interaction and the function of the immune system, and mutated or natural-type Vimentin was involved in the inflammatory responses and the pathogenesis of various autoimmune diseases (Autoimmun Rev. 2018 September: 17 (9): 926-934. doi: 10.1016/j.autrev.2018.04.004. Epub 2018 Jul. 17.), and thus, even in systemic sclerosis, the effect of the STAT6 inhibitor on the expression pattern of cell surface Vimentin (CSV) was also identified. Specifically, the expression of fibrosis factors aSMA, Col1, and IL-17 and CSV-positive cells in the skin tissue obtained in Example 4-1 was identified by immunohistochemical staining.

As a result, as illustrated in FIG. 6, it has been identified that compared to the wild-type mouse, in the Vehicle group, due to increased skin fibrosis, the expression of fibrosis factors aSMA, Col1, and IL-17 in the skin tissue and the number of CSV positive cells were significantly increased, but when the STAT6 inhibitor was administered, the increased expression of aSMA, Col1, and IL-17 and the increased number of CSV positive cells were significantly reduced.

### <5-2> Identification of Expression Inhibition of Fibrosis Factors and Cell Surface Vimentin in Lung Tissue

It was identified whether the STAT6 inhibition of the present disclosure inhibited cell surface Vimentin in the tissue. Specifically, the expression of fibrosis factors aSMA, Col1, and IL-17 and CSV-positive cells in the lung tissue obtained in Example 4-2 was identified by immunohistochemical staining.

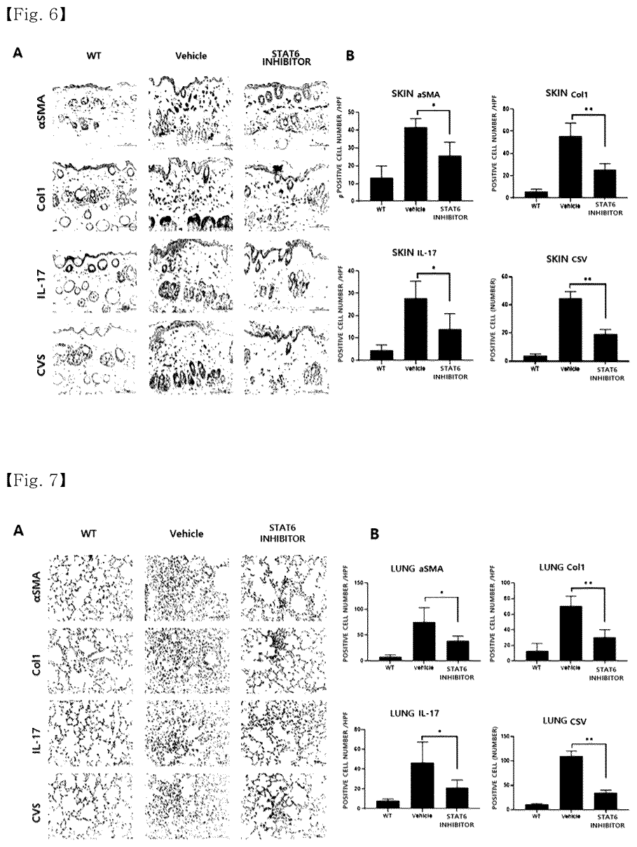

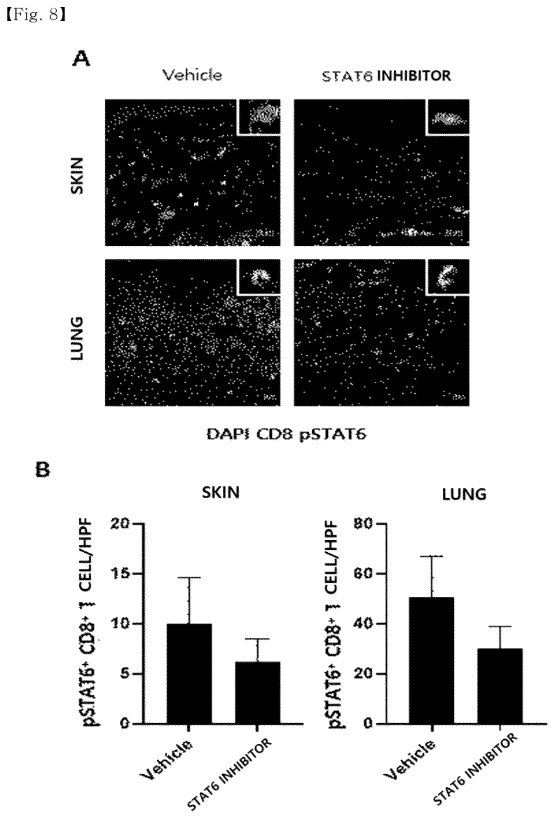

As a result, as illustrated in FIG. 7, it has been identified that compared to the wild-type mouse, in the Vehicle group, due to increased pulmonary fibrosis, the expression of fibrosis factors aSMA, Col1, and IL-17 in the tissue and the number of CSV positive cells were significantly increased, but when the STAT6 inhibitor was administered, the increased expression of fibrosis factors aSMA, Col1, and IL-17 and the increased number of CSV positive cells were significantly reduced.

### <Example 6> Identification of Inhibition of the Expression of STAT6-Expressing CD8 T Cells

In systemic sclerosis, STAT6 was a major transcription factor for a CD8 T cell, and recently, it has been known that in systemic sclerosis, the persistent survival of resident memory CD8 T cells in the tissue increased the fibrosis response and inflammatory response of fibroblasts, and thus it has been identified whether the expression of pSTAT6 positive and CD8 positive T cells, which were active types of STAT6, was controlled by treatment with the STAT6 inhibitor of the present disclosure. Specifically, in the skin and lung tissues obtained in Example 4, the pSTAT6 and CD8 positive T cells were analyzed by confocal microscopy.

As a result, as illustrated in FIG. 8, it has been identified that compared to the Vehicle group which was the systemic sclerosis animal model, in the group treated with the STAT6 inhibitor, the expression of pSTAT6 and CD8 positive T cells in skin and lung tissues was significantly reduced, and thus it has been identified that the expression of active-type STAT6 and CD8 positive T cells was reduced by treatment with the STAT6 inhibitor.

### <Example 7> Identification of the Effect of STAT6 Inhibition in Vimentin Antigen-Specific Systemic Sclerosis Animal Model

### <7-1> Identification of the Effect of Inhibition on Skin and Tissue Fibrosis

In systemic sclerosis, Vimentin increased the disease symptom activity of systemic sclerosis, and thus, the effect of the STAT6 inhibitor was evaluated in a Vimentin antigen-specific systemic sclerosis animal model. Specifically, systemic sclerosis was induced by immunizing a 8-week-old SKG mouse with a Vimentin protein as an antigen and then subcutaneously injecting 100 μL of Bleomycin (BLM) six times a week for two weeks. Thereafter, on week 2 of systemic sclerosis induction, the mouse was secondary immunized with the Vimentin protein, and then subcutaneously injected with the STAT6 inhibitor of the present disclosure three times a week at a concentration of 10 mg/kg until week 5 from the starting of the experiment. Thereafter, at the end point of the experiment, the mouse was humanely sacrificed, and skin and lung tissues were obtained, and then the thickness of the skin dermal layer was identified by H&E staining, and the expression of CSV in the lung tissue was identified by immunohistochemical staining. The specific experimental process was illustrated in FIG. 9, and as a control group, a Vimentin antigen-specific systemic sclerosis animal model (vim SSc group) with increased disease symptom activity by a Vimentin antigen was used.

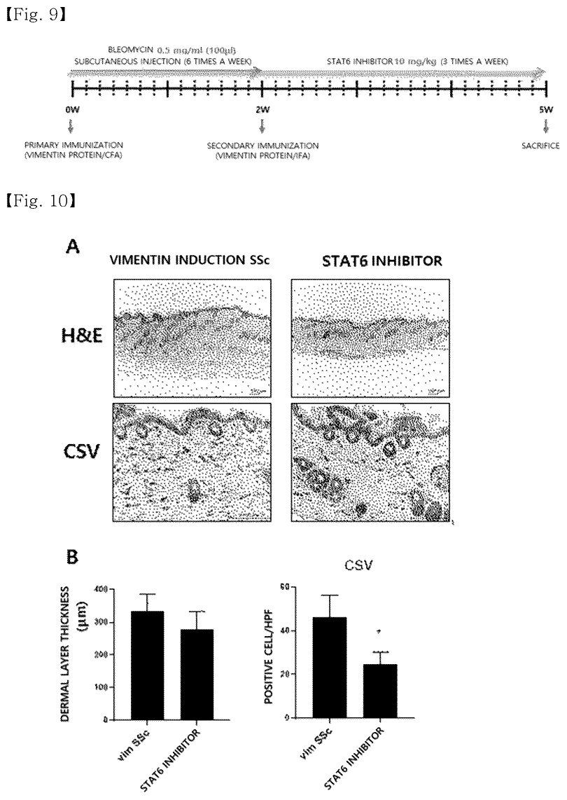

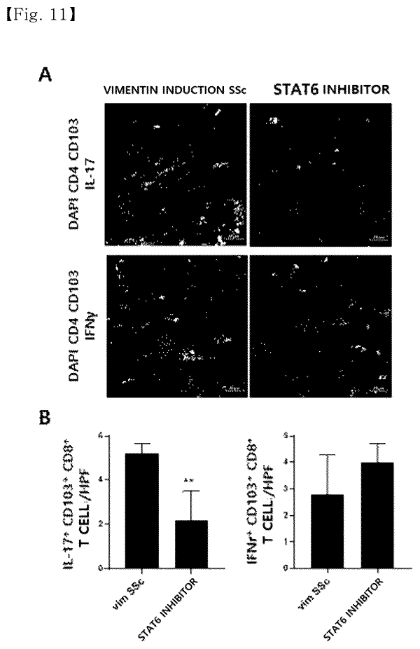

As a result, as illustrated in FIG. 10, it has been identified that compared to the vim SSc group, the thickness of the skin dermis layer was significantly reduced in the group treated with the STAT6 inhibitor. In addition, it has been identified that the expression of CSV in the skin tissue was significantly reduced, and thus it has been identified that the STAT6 inhibitor of the present disclosure may alleviate systemic sclerosis in which the disease symptom activity was increased by the Vimentin antigen.

### <7-2> Identification of Control of IL-17 Expression Cells

In systemic sclerosis, CD8 TRM was known to exist continuously in tissues and accelerate skin inflammation and fibrosis, and thus it has been identified whether the STAT6 inhibitor of the present disclosure inhibited CD8 TRM expression. Specifically, in the skin tissue of the mouse sacrificed in Example 7-1, IL-17 expression CD103 and CD8 positive cells were identified by staining with DAPI, anti CD4 FITC, anti CD103-PE, anti IL-17-APC, and anti IFNγ-APC, and using a confocal microscope. In addition, the CD103 and CD8 positive cells expressing IFNγ were identified.

As a result, as illustrated in FIG. 11, it has been identified that compared to the vim SSc group, in the group treated with STAT6 inhibitor, the IL-17 expression CD103 and CD8 positive cells (CD8 TRM), which were pathogenic cells of systemic sclerosis, were significantly reduced. In addition, there was no change in IFNγ expression CD103 and CD8 positive cells, which were immune regulatory cells.

According to the present disclosure, it has been identified that the STAT6 inhibitor inhibits the expression of a profibrotic T cell and an M2 macrophage which are immunocytes related to systemic sclerosis, and increases the expression of Treg, and inhibits the expression of TGF-β, Col1a1, and α-SMA which are fibrosis factors related to systemic sclerosis. In addition, it has been identified that when a STAT6 inhibitor was administered to an animal model of systemic sclerosis, the STAT6 inhibitor inhibits the activity of an M2 macrophage capable of inducing fibrosis in a mouse blood, and suppresses the thickness of the skin dermis and pulmonary fibrosis. It has been identified that the expression of cell surface Vimentin, which is a Vimentin mutant protein related to systemic sclerosis, is reduced in a skin tissue and a lung tissue, and that the Vimentin mutant protein may be controlled. In addition, the presence of a pSTAT6-expressing CD8 T cell in a fibrosis tissue has been identified, and it has been identified that the expression of a pSTAT6-expressing CD8 T positive cell is controlled via injection of the STAT6 inhibitor. Even in an animal model with increased Vimentin-specific disease symptom activity, the STAT6 inhibitor inhibits antigen-specific tissue fibrosis of systemic sclerosis with activated disease symptoms, and inhibits the expression of IL-17 cytokine-expressing CD8 positive TRM capable of inducing fibrosis and inflammation, which is a pathogenic cell of systemic sclerosis.

1. A method for preventing or treating systemic sclerosis, comprising administering to a subject in need thereof a pharmaceutical composition comprising a signal transducer and activator of transcription 6 (STAT6) inhibitor as an active ingredient.

2. The method of claim 1, wherein the STAT6 inhibitor is a compound represented by Chemical Formula 1 below or a pharmaceutically acceptable salt thereof

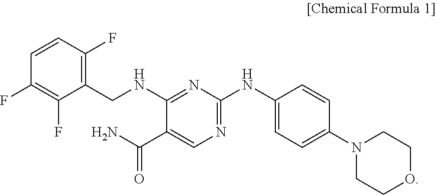 [SMILES: NC(=O)c1cnc(Nc2ccc(N3CCOCC3)cc2)nc1NCc1c(F)ccc(F)c1F]

3. The method of claim 1, wherein the STAT6 inhibitor further comprises a substance that specifically binds to a STAT6 gene to inhibit the gene expression of STAT6, or a substance that specifically binds to a STAT6 protein to inhibit the expression or activity of the STAT6 protein.

4. The method of claim 3, wherein the STAT6 gene expression inhibitor is selected from the group consisting of antisense nucleotide, siRNA, and shRNA that complementarily bind to mRNA of the STAT6 gene.

5. The method of claim 3, wherein the STAT6 protein expression or activity inhibitor is selected from the group consisting of a peptide, a peptidomimetic, a substrate analog, an aptamer and an antibody that complementarily binds to the STAT6 protein.

6. The method of claim 1, wherein the systemic sclerosis further comprises systemic sclerosis with increased disease symptom activity by Vimentin.

7. The method of claim 1, wherein the STAT6 inhibitor reduces a skin thickness.

8. The method of claim 1, wherein the STAT6 inhibitor inhibits tissue fibrosis induced by systemic sclerosis.

9. The method of claim 8, wherein the tissue is a tissue selected from the group consisting of skin, lung, liver, muscle, kidney, intestine and spleen.

10. The method of claim 1, wherein the STAT6 inhibitor controls immunocytes in tissues, spleen or blood, wherein the controlling of the immunocytes reduces the expression of profibrotic T cells or M2 macrophages.

11. (canceled)

12. The method of claim 10, wherein the controlling of the immunocytes is reducing an expression ratio of M2 macrophages and M1 macrophages.

13. The method of claim 10, wherein the controlling of the immunocytes is reducing the activity of M2 macrophages.

14. The method of claim 10, wherein the controlling of the immunocytes is increasing the expression of Treg.

15. The method of claim 10, wherein the controlling of the immunocytes is reducing the expression of pSTAT6 positive and CD8 positive cells; or IL-17 positive, CD103 positive and CD8 positive cells.

16. The method of claim 1, wherein the STAT6 inhibitor reduces fibrosis factors, wherein the fibrosis factors are factors selected from the group consisting of TGF-β, Col1a1, α-SMA and IL-17.

17. (canceled)

18. The method of claim 1, wherein the STAT6 inhibitor inhibits the activity of a Vimentin mutant protein in the tissue.

19. The method of claim 18, wherein the Vimentin mutant protein is cell surface Vimentin.

20. A method for preventing or alleviating systemic sclerosis, comprising a signal transducer and activator of transcription 6 (STAT6) inhibitor as an active ingredient.

21. A quasi-drug composition for preventing or alleviating systemic sclerosis, comprising a signal transducer and activator of transcription 6 (STAT6) inhibitor as an active ingredient.

22-23. (canceled)

24. A method of treating fibrosis by a Vimentin mutant protein, comprising administering a signal transducer and activator of transcription 6 (STAT6) inhibitor in a pharmaceutically effective amount to a subject in need thereof.
